(19) United States
(12) Patent Application Publication
(10) Pub. No.: US 2005/0119283 A1
(43) Pub. Date: Jun. 2, 2005

(54) Treatment of circadian rhythm disorders with NPY Y5 receptor antagonist

(75) Inventors: Francisca Fatima Matos, Mystic, CT (US); Jeffrey S. Sprouse, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/951,325

(22) Filed: Sep. 27, 2004

Related U.S. Application Data
(60) Provisional application No. 60/506,246, filed on Sep. 26, 2003.

(51) Int. Cl. A61K 31/52; A61K 31/4747

(52) U.S. Cl. 514/263.2; 514/278

(57) ABSTRACT

A method for treating circadian rhythm disorders in mammals comprising administering to a mammal an effective amount of an NPY Y5 receptor antagonist. In particular, a method is provided for enhancing the effects of light on circadian rhythm.

### BACKGROUND OF THE INVENTION

This invention relates to a method for treating circadian rhythm disorders in mammals. The term “circadian rhythm disorders”, as used herein, is defined as a disorder related to a disruption in any circadian rhythm in which there exists poor rhythm synchrony to environmental cues. In particular, this invention relates to a method of enhancing the effects of light on circadian rhythms and/or increasing the amplitudes of these rhythms in mammals comprising administering to a mammal an effective amount of an NPY Y5 receptor antagonist.

Circadian rhythms are cyclical patterns of animal behavior which are synchronized with environmental cycles of day and night and occur on a 24-hour time scale. Exposure to light is a key factor. Associated with these rhythms are changes of great physiological importance including but not limited to hormone synthesis and release, body temperature, cardiovascular function, sleep and activity cycles. It is believed that a single mechanism, a molecular clock, regulates these circadian rhythms in multicellular animals. The term “molecular clock”, as used herein, is defined as the cellular timing mechanism in which a sequence of events at the molecular level (gene transcription and protein synthesis) repeats itself on a 24-hour basis and accounts for the oscillation of the rhythms and resultant cyclical patterns of animal behavior. The term “circadian clock”, as used herein, is defined as the biological mechanism that accounts for the rhythmic nature of such physiological functions and is used interchangeably with the term “biological clock”.

Modern patterns of living and technology including jet travel (jet lag), especially between time zones; artificial light; and shift work hours may be poorly synchronized with internal circadian clocks. As a consequence of these modern schedules, performance degradation may manifest in loss of manual dexterity, reflexes, memory, winter depression, and general fatigue derived from lack of sleep.

Examples of disorders and conditions associated with circadian rhythms are depression, unipolar depression, bipolar disorder, seasonal affective disorder, dysthymia, anxiety, schizophrenia, Alzheimers Disease, rapid eye movement (REM) sleep disorders, advanced sleep phase syndrome, delayed sleep phase syndrome, non-24-hour sleep-wake disorder, hypersomnia, parasomnia, narcolepsy, nocturnal enuresis, obesity and restless-leg syndrome.

It is known that in humans, melatonin levels appear to be regulated by the circadian clock. Melatonin levels have been observed to rise and fall with sleep and wakefulness.

Attempts to control circadian rhythm key markers with therapeutic doses of melatonin are disclosed in United States Patent Application Publication No. 2003/0008912, which was published on Jan. 9, 2003.

The use of nitric oxide synthase (NOS) inhibitors either alone or in combination with a selective serotonin reuptake inhibitor (SSR1) in the treatment of circadian rhythm disorders is disclosed in WO 00/71107.

Melatonin activity in the regulation of the circadian clock is transmitted by certain pharmacologically specific, high affinity receptors. U.S. Pat. No. 6,037,131 discloses the use of DNA receptor genes as promoter regions for high-affinity melatonin receptors.

U.S. Pat. No. 5,703,239 discloses the use of indanyl-substituted piperidines as useful melatonergic agents in the treatment of anxiety, depression and various central nervous system (CNS) disorders related to circadian rhythms.

Neuropeptide Y (NPY), a 36 amino acid peptide neurotransmitter, is a member of the pancreatic class of neurotransmitters/neurohormones which has been shown to be present in the CNS and mediate biological responses via NPY specific receptors (e.g. Y1, Y2, Y5 receptors).

In laboratory animal studies, NPY significantly affects the natural ability of light to shift the timed cycles of circadian rhythms. Specifically, daytime phase-shifting, manifested as an advance of the occurrence of the normal rhythm, is mediated through the NPY Y2 receptor. NPY Y1/Y5 and Y5 receptors have been shown to be related to nighttime phase-shifting effects (Yannielli et al J. Neurosci. 2001 (14): 5367-73).

U.S. Pat. No. 6,514,966 discloses the use of NPY Y5 antagonists for the treatment of obesity and related feeding disorders.

WO 99/01128 discloses certain NPY Y5 receptor mediators useful in treating feeding disorders as well as certain cardiovascular diseases.

WO 03/051356 proposes selected NPY Y5 antagonists for blocking the phase-shifting effects of light in a mammal.

The foregoing patents and patent application are incorporated by reference herein in their entirety.

### SUMMARY OF THE INVENTION

This invention provides a method of modulating circadian rhythm responses to light in a mammal by administering to a mammal an amount of an NPY Y5 receptor antagonist effective in modulating circadian rhythm responses to light.

This invention further provides a method for enhancing the effects of light on circadian rhythm in a mammal by administering a light enhancing amount of an NYP Y5 receptor antagonist to a mammal including humans.

In another embodiment of the present invention circadian rhythm modulation; and, more specifically, enhancement of the effects of light on circadian rhythm in a mammal are achieved by administering to a mammal an effective amount of an NYP Y5 receptor antagonist having the formula 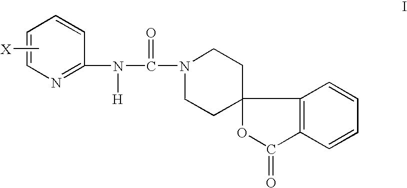
or a pharmaceutically acceptable salt, solvate or prodrug thereof or of any of the foregoing,
wherein X is selected from the group consisting of chlorine, bromine, iodine, trifluoromethyl, hydrogen, cyano, C₁ to C₆ alkyl, C₁ to C₆ alkoxy, C₅ or C₆ cycloalkyl, ester, amido, aryl and heteroaryl.

In a preferred embodiment, the NPY Y5 antagonist is a compound of formula 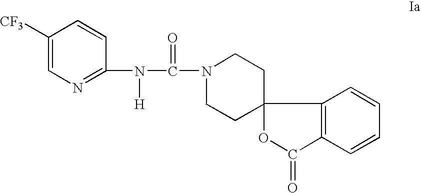
or a pharmaceutically acceptable salt, solvate or prodrug thereof or of any of the foregoing.

In another embodiment of the present invention a method of modulating circadian rhythm responses; and, specifically, a method of enhancing the effects of light on circadian rhythm responses in a mammal is provided comprising the administering of a compound of the formula 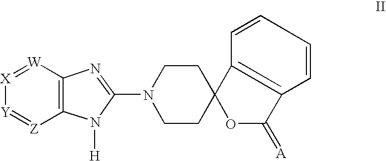
or a pharmaceutically acceptable salt, solvate or prodrug thereof or any of the foregoing; wherein A is oxygen or hydrogen; wherein W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono- or di-(C₁-C₆)alkylamino or (C₁-C₆)alkoxy, (C₃-C₇)cycloalkyl, (C₃-C₇)cycloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono- and di(C₁-C₆)alkylamino(C₁-C₆)alkyl.

The term “enhancement of the effects of light an circadian rhythm” refers to the ability of compounds of formula I and II to reverse the blockage caused by NPY on the phase advancing effect of light on circadian rhythm in a mammal.

In a preferred embodiment, the compound of formula II is a compound having the formula

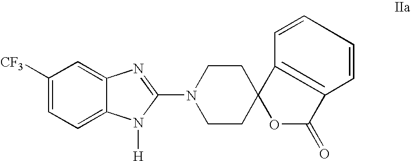

This invention provides a method of treating circadian rhythm disorders in mammals including humans by administering to a mammal an amount of an NPY Y5 receptor antagonist that is effective in blocking the effects of NPY on the circadian clock.

In one embodiment of the above recited method for treating circadian rhythm disorders, the NPY Y5 receptor antagonist is administered to a mammal prior to experiencing circadian rhythm disorders.

In another embodiment of the above recited method, the NPY Y5 antagonist is administered to a mammal predisposed to or at risk of experiencing circadian rhythm disorders.

This invention also provides a method for treating circadian rhythm disorders in a mammal by administering to a mammal an amount of an NPY Y5 antagonist wherein the antagonist is a compound of formula 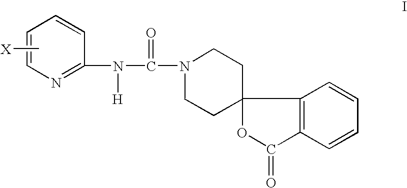
or a pharmaceutically acceptable salt, solvate or prodrug thereof or of any of the foregoing,
wherein X is selected from the group consisting of chlorine, bromine, iodine, trifluoromethyl, hydrogen, cyano, C₁ to C₆ alkyl, C₁ to C₆ alkoxy, C₅ or C₆ cycloalkyl, ester, amido, aryl and heteroaryl.

In a preferred embodiment, the NPY Y5 antagonist is a compound of formula 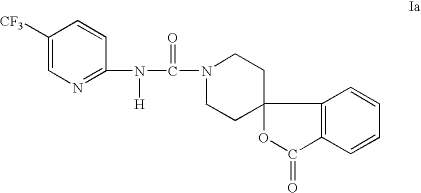
or a pharmaceutically acceptable salt, solvate or prodrug thereof or of any of the foregoing,

This invention further provides a method for treating circadian rhythm disorders in a mammal by administering to a mammal an amount of an NPY Y5 antagonist wherein the antagonist is a compound of formula 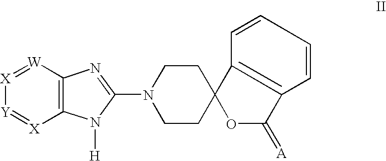
or a pharmaceutically acceptable salt, solvate or prodrug thereof or any of the foregoing; wherein A is oxygen or hydrogen; wherein W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono- or di-(C₁-C₆)alkylamino or (C₁-C₆)alkoxy, (C₃-C₇)cycloalkyl, (C₃-C₇)cycloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono- and di(C₁-C₆)alkylamino(C₁-C₆)alkyl.

In a preferred embodiment, the NPY Y5 antagonist is a compound of the formula 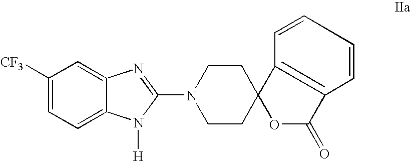
or a pharmaceutically acceptable salt, solvate or prodrug thereof or of any of the foregoing.

For compounds having asymmetric centers, all optical isomers, racemates and mixtures thereof are encompassed in the present invention.

Where a compound exists in various tautomeric forms, the invention is not limited to any one of the specific tautomers.

This invention is based on the discovery that the NPY-caused blockade of light induced shifting of circadian cycles (phase advances or phase delays) can be reversed by the NPY Y5 receptor antagonists and the discovery that by themselves NPY Y5 antagonists enhance the shifting of circadian rhythms by light. For purposes of the present invention, the term “NMDA-induced” refers to an in vitro procedure for simulating the phase shifting effects of natural light by the application of N-methyl-D-aspartate (NMDA) to brain tissue preparations.

In one embodiment of the present invention, a method is provided for modulating circadian rhythm responses to light in a mammal by administering to a mammal a compound of formula I or formula II; preferably said compound is of formula Ia or IIa.

In another embodiment the modulating of circadian rhythm responses comprises phase-shifting, resetting of the circadian clock and enhancing the rate of re-entrainment.

As used herein the term “modulating” refers to a regulation of the observed blockade caused by NPY and/or a regulation of the phase shifting effects of light. Modulation of circadian rhythm responses includes phase-shifting, resetting of the circadian clock, enhancing the rate of re-entrainment, and changes in the amplitude of circadian rhythm. As used herein the term “resetting of the circadian clock” refers to any action which corrects the phase and/or amplitude of the circadian rhythm resulting from modern patterns of daily living and/or a biological abnormality in brain function to one properly synchronized with the phase of solar day.

The term “enhancing the rate of re-entrainment” refers to any action that decreases the amount of time required to adjust the internal biological clock to the prevailing phase of the solar day.

“Phase-shifting” encompasses both phase advances and phase delays. “Phase advance” refers to a shift in the pattern of circadian rhythm to an earlier point in time. “Phase delay” refers to a shift in the pattern of circadian rhythm to a later point in time.

As used herein the term “amplitude of circadian rhythm” refers to the difference between the lowest level of activity for a given biological activity tied to a circadian rhythm to the highest level of said activity as illustrated in FIG. 1 for neuronal firing rates.

In particular, the invention comprises a method for reversing NPY caused blockade by the administration of NPY-Y5 antagonist compounds of Formula I and Formula II. Preferably the NPY-Y5 antagonist is a compound of Formula Ia or Formula IIa. Additionally, the invention comprises a method for enhancing the effects of light on circadian phase shifting.

Evidence of NPY-Y5 caused blockade for compounds of Formula I and Formula II was obtained in the in vitro and in vivo methods described below.

In a preferred embodiment, the compound of Formula Ia exhibits, in vitro, about 70% reversal of the blockade caused by NPY and the compound of Formula IIa exhibits about 95% reversal of the blockade caused by NPY.

In another preferred embodiment, the compound of Formula IIa exhibits, in vivo, about 90% reversal of the blockade caused by NPY.

In yet another embodiment, the compound of Formula IIa, in the absence of NPY, enhances, in vivo, the light induced phase shift by 160% of that achieved by light alone.

In another embodiment, the invention includes a method for reversing the effects of NPY on the light induced phase advances in a mammal comprising administering to said mammal an effective amount of a compound of Formula I or Formula II to reverse the effect of NPY.

In another embodiment of the present invention a method is provided for treating circadian rhythm disorders comprising administering to a mammal in need of such treatment a therapeutically effective amount of a compound which provides a blockade of at least 70% to NPY Y5 receptors. Preferably said compound is a compound of formula I or formula II and most preferably of formula Ia or formula IIa.

The present invention also comprises a method of treating circadian rhythm phase disorders comprising administering to a mammal in need of such treatment a therapeutically effective amount of a compound which effectively blocks NPY Y5 receptor sites. Preferably the compound is selected from the group consisting of compounds of Formula I and Formula II and most preferably the compound is selected from Formula Ia and Formula IIa.

Circadian rhythm disorders are comprised of disorders related to modern patterns of living and to biological abnormalities in brain function. Those disorders contemplated for treatment by the present invention include disorders of phase related to jet lag and shift work, depression, unipolar depression, bipolar disorder, seasonal affective disorder, dysthymia, anxiety, schizophrenia, Alzheimers Disease, rapid eye movement (REM) sleep disorders, advanced sleep phase syndrome, delayed sleep phase syndrome, non-24-hour sleep-wake disorder, hypersomnia, parasomnia, narcolepsy, nocturnal enuresis, obesity and restless-leg syndrome.

In one embodiment, a method is provided which enhances an in vivo light induced phase shifts by 200% of that achieved by light alone.

In another embodiment, the present invention provides a method of treating circadian rhythm disorders in mammals including humans comprising administering to a mammal a light enhancing amount of an NPY Y5 antagonist effective in treating circadian rhythm disorders.

In another embodiment, the present invention provides a method of treating circadian rhythm disorders comprising circadian rhythm phase-shift disorders. Preferably said phase shift disorders include phase shift advances or phase shift delays.

In another embodiment, circadian rhythm disorders are comprised of changes in the amplitude of the circadian rhythm.

### BRIEF DESCRIPTION OF THE DRAWING

FIG. 1 is a graphic illustration of the terms used herein.

Brain slices containing the SCN were taken on a nominally “preparatory day”. During the subsequent night, at 3-3.5 hours before the scheduled onset of light, drugs were administered to the bath. Neuronal recordings were made beginning early the next day, nominally the “experimental day”, and continued until the peak firing rate could be established. A shift in this peak to an earlier point in time is referred to as a “phase advance”.

### DETAILED DESCRIPTION OF THE INVENTION

The compounds of Formula I and Formula II can be prepared by the synthetic methods described and referred to in WO 02/48152 which is hereby incorporated by reference herein in its entirety.

Representative compounds of Formula I include, but are not limited to:
1′-(4-t-butyl-pyridylcarbamoyl)-spiroisobenzofuran-1,4′-piperidine-3-one;
1′-(4-isopropyl-pyridylcarbamoyl)-spiroisobenzofuran-1,4′-piperidine-3-one;
1′-(4-trifluoromethyl-pyridylcarbamoyl)-spiroisobenzofuran-1,4′piperdine-3-one;
and their pharmaceutically acceptable salts.

Representative compounds of Formula II include but are not limited to:
1′-(1H-benzimidazol-2-yl)-spiro[isobenzofuran-1,4′-piperidin]-3-one;
1′-(5-cyano-1H-benzimidazol-2-yl)-spiro[isobenzofuran-1,4′-piperidin]-3-one;
1′-(5-acetyl-1H-benzimidazol-2-yl)-spiro[isobenzofuran-1,4′-piperidin]-3-one;
1′-(5-carboxy-1H-benzimidazol-2-yl)-spiro[isobenzofuran-1,4′-piperidin]-3-one methyl ester;
1′-(5′-pyridin-3-yl-1H-benzimidazol-2-yl)-spiro[isobenzofuran-1,4′-piperidin]-3-one;
1′-(5-methyl-1H-benzimidazol-2-yl)-spiro[isobenzofuran-1,4′-piperidin]-3-one;
1′-(5-methoxy-1H-benzimidazol-2-yl)-spiro[isobenzofuran-1,4′-piperidin]-3-one;
1′-(5-chloro-1H-benzimidazol-2-yl)-spiro[isobenzofuran-1,4′-piperidin]-3-one;
1′-(5-fluoro-1H-benzimidazol-2-yl)-spiro[isobenzofuran-1,4′-piperidin]-3-one; and
1′-(5-trifluoromethyl-1H-benzimidazol-2-yl)-spiro[isobenzofuran-1,4′-piperidin]-3-one;
1′-(6-trifluoromethy-3-H-imidazo[4,5-b]pyridine-2-yl)-spiro[isobenzofuran-1,4′-piperidin]-3-one;
1′-(7-chloro-1H-benzimidazol-2-yl)-spiro[isobenzofuran-1,4′-piperidin]-3-one;
1′-(1H-benzimidazol-2-yl)-spiro[isobenzofuran-1,4′-piperidin]-3-one;
1′-(5-n-propylsulfonyl-1H-benzimidazol-2-yl)-spiro[isobenzofurarn-1,4′-piperidin]-3-one;
1′-(5 cyano-1H-benzimidazol-2-yl)-spiro[isobenzofuran-1,4′-piperidin]-3-one;
1′-(5-acetyl-1H-benzimidazol-2-yl)-spiro[isobenzofuran-1,4′-piperidin]-3-one;
1′-(5-carboxy-1H-benzimidazol-2-yl)-spiro[isobenzofuran-1,4′-piperidin]-3-one, methyl ester;
1′-(5′pyrazin-2-yl-1H-benzimidazol-2-yl)-spiro[isobenzofuran-1,4′-piperidin]-3-one;
1′-(5′pyridin-3-yl-1H-benzimidazol-2-yl)-spiro[isobenzofuran-1,4′-piperidin]-3-one;
1′-(5-trifluoromethoxy-1H-benzimidazol-2-yl)-spiro[isobenzofuran-1,4′-piperidin]-3-one;
1′-(5-methyl-1H-benzimidazol-2-yl)-spiro[isobenzofuran-1,4′-piperidin]-3-one;
1′-(5-benzoyl-1H-benzimidazol-2-yl)spiro[isobenzofuran-1,4′-piperidin]-3-one;
1′-(5-methoxy-1H-benzimidazol-2-yl)-spiro[isobenzofuran-1,4′-piperidin]-3-one;
1′-(5-chloro-1H-benzimidazol-2-yl)-spiro[isobenzofuran-1,4′-piperidin]-3-one;
6-bromo-7-chloro-2-(spiro[isobenzofuran-1,4′-piperidin]-3-one-3H-imidazo[4,5-b]pyridine;
1′-(5-fluoro-1H-benzimidazol-2-yl)-spiro[isobenzofuran-1,4′-piperidin]-3-one;
1′-(5-methyl-1H-benzimidazol-2-yl)-spiro[isobenzofuran-1,4′-piperidin]-3-one;
1′-(5-methylsulfonyl-1H-benzimidazol-2-yl)-spiro[isobenzofuran-1,4′-piperidin]-3-one;
1′-(5-oxazol-2-yl-1H-benzimidazol-2-yl)-spiro[isobenzofuran-1,4′-piperidin]-3-one;
1′-(5,6-difluoro-1H-benzimidazol-2-yl)-spiro[isobenzofuran-1,4′-piperidin]-3-one;
1′-(5-phenyl-1H-imidazo[4,5-b]pyrazin-2-yl)-spiro[isobenzofuran-1,4′-piperidin]-3-one;
1′-(5-trifluoromethyl-1H-benzimidazol-2-yl)-spiro[isobenzofuran-1,4′-piperidin]-3-one;
1′-(5,7-dichloro-1H-benzimidazol-2-yl)-spiro[isobenzofuran-1,4′-piperidin]-3-one;
1′-(5,6-dimethoxy-1H-benzimidazol-2-yl)-spiro[isobenzofuran-1,4′-piperidin]-3-one;
1′-(5-trifluoromethylsulfonyl-1H-benzimidazol-2-yl)-spiro[isobenzofuran-1,4′-piperidin]-3-one;
1′-(5-(3,5-dime:thyi-isoxazol-4-yl)-1H-benzimidazol-2-yl)-spiro[isobenzofuran-1,4′-piperidin]-3-one;
1′-(5-ethoxy-1H-benzimidazol-2-yl)-spiro[isobenzofuran-1,4′-piporidin]-3-one; and
5-chloro-2-(spiro[isobenzofuran-1,4′-piperidin]-3-one-3H-imidazo[4,5-b]pyridine; and their pharmaceutically acceptable salts.

The compounds of Formula I and II which are basic in nature are capable of forming a wide variety of different salts with various inorganic and organic acids. Although such salts must be pharmaceutically acceptable for administration to animals, it is often desirable in practice to initially isolate a compound of the Formula I and II from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, e.g. salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate, i.e., 1,1′-methylene-bis-(2-hydroxy-3-naphthoate), salts.

The compounds of Formula I and II may advantageously be used in conjunction with one or more other therapeutic agents, for instance, different antidepressant agents such as tricyclic antidepressants (e.g. amitriptyline, dothiepin, doxepin, trimipramine, butripyline, clomipramine, desipramine, imipramine, iprindole, lofepramine, nortriptyline or protriptyline), monoamine oxidase inhibitors (e.g. isocarboxazid, pheneizine or tranylcyclopramine) or 5-HT re-uptake inhibitors (e.g. fluvoxamine, sertraline, fluoxetine or paroxetine). It may also be used with acetocholinesterases such as donepezil. It is to be understood that the present invention covers the use of a compound of Formula I and II or a physiologically acceptable salt or solvate thereof in combination with one or more other therapeutic agents.

The compounds of the invention are generally administered as pharmaceutical compositions in which the active principle is mixed with a pharmaceutical excipient or carrier. The active compound or principle may be formulated for oral, buccal, intramuscular, parenteral (e.g. intravenous, intramuscular or subcutaneous) or rectal administration or in a form suitable for administration by inhalation or insufflation.

Suitable forms of oral administration include tablets, capsules, powders, granules and oral solutions or suspensions, sublingual and buccal forms of administration.

When a solid composition is prepared in tablet form, the main excipient is mixed with a pharmaceutical excipient such as gelatin, starch, lactose, magnesium stearate, talc or gem arabic. Tablets may be coated with a suitable substance like sugar so that a given quantity of the active compound is released over a prolonged period of time.

Liquid preparations for oral administration may be in the form of a solution, syrup, or suspension. Such liquids may be prepared by conventional methods using pharmaceutically acceptable ingredients such as suspending agents (e.g. sorbitol syrup); emulsifying agents (e.g. lecithin); non-aqueous vehicles (e.g. ethyl alcohol); and preservatives (e.g. sorbic acid).

Formulations for parenteral administration by injection or a infusion may be presented in unit dosage form e.g. in ampules in the form of solutions or emulsions in oily or aqueous vehicles.

The compositions may also be formulated in rectal formulations such as suppositories or retention enemas.

For intranasal or inhalation administration, the compounds are delivered in the form of a solution or suspension from a pump spray or a container pressurized with suitable propellant.

In connection with the use of compounds of Formulas I or II it is to be noted that these compounds may be administered either alone or in combination with a pharmaceutically acceptable carrier. Such administration may be carried out in single or multiple doses. More particularly the composition may be combined with various pharmaceutically acceptable inert carriers in the form of tablets, capsules, lozenges, hard candies, powders, syrup, aqueous suspension, injectable solutions, elixirs, syrups, and the like.

A proposed dose of the active compounds of the invention for oral, parenteral or buccal administration to the average adult human for the treatment of the conditions referred to above (e.g. depression) is about 0.1 to about 200 mg of the active ingredient per unit dose which could be administered, for example, 1 to 4 times per day.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

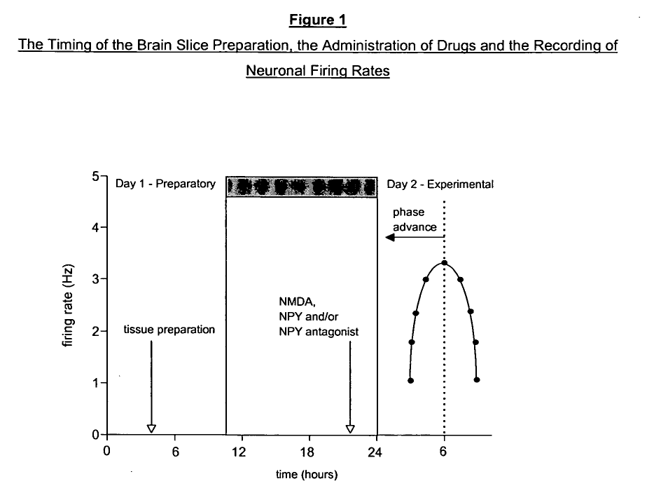

Biological activity of the NPY Y5 antagonist compounds of the present invention was determined is a series of in vitro and in vivo laboratory experiments described herein below. In laboratory animals, antagonists of the NPY Y5 receptor blocked the ability of exogenously applied NPY to reduce the phase advance produced by exposure to light. NPY Y5 antagonists, in the absence of exogenous NPY, also significantly improved the natural ability of light to produce a phase advance. The term “phase advance”, as used herein, is defined as a shift in the pattern of circadian rhythm to an earlier point in time and is illustrated in FIG. 1.

### EXAMPLES

Phase advances were measured in vitro by sampling spontaneous activity from neurons in a brain slice preparation of the suprachiasmatic nucleus, herein abbreviated SCN, that is known to contain the circadian clock. The term “brain slice preparation”, as used herein, is defined as a cut section of brain that is placed in a plastic chamber and kept fully functioning by providing it with ACSF (artificial cerebrospinal fluid) that has been warmed and infused with oxygen. Recordings of the spontaneous activity of neurons in the SCN brain slice preparation follow a 24-hour pattern of activity that marks the circadian rhythm. Following application of N-methyl-D-aspartate (NMDA), a compound that mediates the phase advances elicited by light in vivo, neurons in the SCN shift their pattern of firing in vitro to reflect a phase advance. Application of NPY blocks the phase advances elicited by NMDA; NPY Y5 antagonists of formula Ia and IIa block these effects of NPY.

### 1. In Vitro

Animals and tissue preparation. Male golden hamsters (LVG, Charles River, 40-60 days old) were housed under a light:dark schedule of 14 hours of constant light and 10 hours of constant dark, with food and water available ad libitum. Hamsters were administered an overdose of halothane anesthesia and decapitated during the subjective day. Hypothalamic slices (500 μm) containing the suprachiasmatic nucleus (SCN) were placed in a gas-fluid interface slice chamber (Medical Systems BSC with Haas top), continuously bathed (1 ml/min) in artificial cerebrospinal fluid (ACSF) containing 125.2 mM NaCl, 3.8 mM KCl, 1.2 mM KH2PO4, 1.8 mM CaCl2, 1 mM MgSO4, 24.8 mM NaHCO3, 10 mM glucose. ACSF (pH 7.4) was supplemented with an antibiotic (gentamicin, 50 mg/l) and a fungicide (amphotericin, 2 mg/l) and maintained at 34.5° C. Warm, humidified 95% oxygen:5% carbon dioxide was continuously provided to the slice preparation.

Electrophysiological studies. Extracellular single unit activity of SCN cells was detected with glass micropipette electrodes filled with ACSF, advanced through the slice using a hydraulic microdrive. The signal was further amplified and filtered, and was continuously monitored by an oscilloscope and audio monitor. Firing rate was analyzed using data acquisition software and a customized program for calculation of descriptive statistics. The term “firing rate”, as used herein, is defined as the rate at which the neurons produce an action potential during the period of recording and is indicative of their level of functioning. Firing rates in the range of 1 to 10 Hz are typical for SCN neurons. A number of experiments in each condition were recorded “blind” where the person recording data had no knowledge of the treatment. One slice was recorded from each animal. A total number of 42 slices was recorded.

Data analysis. Data were initially grouped into 1 h bins and an analysis of variance test was used to determine if any bins differed from the others. If the analysis of variance test indicated significant differences, data were smoothed using 1 h running means with a 15-minute lag. The time of the middle of the 1 h bin with the highest mean firing rate after processing by this smoother was taken as the time of peak firing rate for that slice. Phase advances of individual slices were measured relative to the average time of peak firing of control slices. Significant differences between groups (p<0.05) were determined by ANOVA followed by Bonferroni method (for all vs control comparisons). Means are reported±standard error.

Results. Control experiments were conducted to determine the time of peak firing rate in SCN brain slices given no drug treatment (Table 1). A phase advance in the time of peak firing was observed in slices given NMDA to mimic the effects of light in the late subjective night, in these experiments, 3.5 hours before lights would be scheduled to come on in the animal quarters. Slices treated with application of NPY 5 min after the NMDA application demonstrated a peak in firing rate at a time similar to that observed in the untreated slices, indicating no phase shift. Thus, this work confirms that NPY blocks the phase advance elicited by NMDA.

NPY Y5 antagonists, compounds of Formula Ia and IIa, were applied at a concentration of 10 μM in the ACSF bathing the slice for 60 min centered on the time of the applications of NMDA and NPY. Application of the antagonist alone did not induce a shift in the phase of spontaneous firing rate. The efficacy of antagonists Ia and Ib are summarized in Table 1 below. Both antagonists were able to prevent NPY from blocking the NMDA-induced phase shift, as is indicated by a peak in firing rate at the advanced phase comparable to experiments with NMDA alone.

A selected NPY Y1 receptor antagonist did not alter the phase resetting action of NMDA, nor did it alter the effect of NPY on the NMDA-induced phase advance.

TABLE 1
EFFECTS OF NPY Y5 ANTAGONISTS ON NMDA-INDUCED
PHASE ADVANCES OF NEURONAL FIRING IN HAMSTER
SCN SLICES MAINTAINED IN VITRO.

| Treatment | | Phase shift (h) |
|---|---|---|
| a. | Control | 0.00 ± 0.17 |
| b. | NPY | −0.18 ± 0.17 |
| c. | NMDA | 2.89 ± 0.08 |
| d. | NMDA + NPY | −0.07 ± 0.09 |
| e. | NMDA + NPY + Formula Ia | 2.03 ± 0.88 |
| f. | Formula Ia | 0.32 ± 0.35 |
| g. | NMDA + NPY + Formula IIa | 2.73 ± 0.16 |
| h. | Formula IIa | 0.07 ± 0.07 |

Experimental Conditions:

Phase advances (h) were calculated as the difference in the occurrence of peak neuronal firing rates of the drug-treated slices relative to control (0.00 h). Means±S.E.M. for N=2−6.
a. Control experiments in which no drug is given and the peak of neuronal firing rate is termed 0 hours.
b. NPY alone experiment in which NPY is given in a bath application 3.5 hours before the scheduled beginning of the animals' period of normal light. The dose of NPY is 2 ng/ml in ACSF delivered by syringe in a single drop (200 nl). There is no effect on the phase of neuronal firing compared to the control experiment.
c. NMDA alone experiment in which NMDA is given in a bath application 3.5 hours before the scheduled beginning of the animals' period of normal light. The dose of NMDA is 100 μM in ACSF delivered by syringe in a single drop (200 nl). There is a resulting phase advance of 2.89 h.
d. NMDA+NPY experiment in which NMDA and NPY are given in a bath application 3.5 hours before the scheduled beginning of the animals' period of normal light. The dose of NMDA is 100 μM in ACSF delivered by syringe in a single drop (200 nl). The dose of NPY is 2 ng/ml in ACSF delivered by syringe in a single drop (200 nl). The dose of NPY precedes the NMDA dose by 5 minutes. There is a complete blockade of the NMDA-induced phase advance by NPY.
e. NMDA+NPY+formula Ia experiment in which NMDA and NPY and NPY Y5 antagonist of formula Ia are given in a bath application 3.5 hours before the scheduled beginning of the animals' period of normal light. The dose of NMDA is 100 μM in ACSF delivered by syringe in a single drop (200 nl). The dose of NPY is 2 ng/ml in ACSF delivered by syringe in a single drop (200 nl). The dose of NPY precedes the NMDA dose by 5 minutes. The dose of NPY Y5 antagonist of formula Ia is 10 μM in ACSF applied in a 60 minute bath application centered on the time of applications for NMDA and NPY. There is a reversal of the effect of NPY on NMDA-induced phase advances by the NPY Y5 antagonist of formula Ia to 70% of the NMDA alone experiment.
f. NPY Y5 antagonist of formula Ia alone experiment in which compound of formula Ia is given in a bath application 3.5 hours before the scheduled beginning of the animals' period of normal light. The dose of NPY Y5 antagonist of formula Ia is 10 μM in ACSF applied in a 60 minute bath application. There is no effect on the phase of neuronal firing compared to the control experiment.
g. NMDA+NPY+formula IIa experiment in which NMDA and NPY and NPY Y5 antagonist of formula IIa are given in a bath application 3.5 hours before the scheduled beginning of the animals' period of normal light. The dose of NMDA is 100 μM in ACSF delivered by syringe in a single drop (200 nl). The dose of NPY is 2 ng/ml in ACSF delivered by syringe in a single drop (200 nl). The dose of NPY precedes the NMDA dose by 5 minutes. The dose of NPY Y5 antagonist of formula IIa is 10 μM in ACSF applied in a 60 minute bath application centered on the time of applications for NMDA and NPY. There is a reversal of the effect of NPY on NMDA-induced phase advances by the NPY Y5 antagonist of formula IIa to 95% of the NMDA alone experiment.
h. NPY Y5 antagonist of formula IIa alone experiment in which compound of formula Ia is given in a bath application 3.5 hours before the scheduled beginning of the animals' period of normal light. The dose of NPY Y5 antagonist of formula IIa is 10 μM in ACSF applied in a 60 minute bath application. There is no effect on the phase of neuronal firing compared to the control experiment.

### 2. In Vivo

The in vivo experimental design included recording a behavioral overt rhythm such as running-wheel activity and exposing the animals to an amount of light that is known to produce a phase advance in this pattern of activity. The term “running-wheel activity”, as used herein, is defined as physical activity measured as revolutions of a wheel permanently positioned in the animals' cages and rotated as the animals run in them. The onset of such behavior is a well regarded marker of timing in circadian rhythms. Application of NPY through a cannula aimed directly into the SCN blocks the ability of light to produce a phase advance; NPY Y5 antagonists of formula IIa block these effects of NPY. Furthermore, when given in the absence of NPY, NPY Y5 antagonists of formula IIa enhance the ability of light to produce phase advances.

Surgery. For in vivo treatment, hamsters (80-100 g) were deeply anesthetized with nembutal (80 mg/kg, i.p.), administered an analgesic (buprenorphine, 0.05 mg/kg, s.c.) and mounted in a stereotaxic instrument in order to rigidly fix the skull. They were surgically implanted with a 25 gauge stainless steel guide cannula aimed at the SCN. After a week of recovery under LD 14:10 (14 hours of light, 10 hours of dark), animals were individually transferred to cages (48×27×20 cm) equipped with wheels. Wheel running activity was recorded with ClockLab hardware and software (Actimetrics, Evanston, Ill.).

Drugs and routes of administration. Animals were briefly anesthetized in order to minimize the stress induced by restraint during cannula injections with a mixture of oxygen and isoflurane administered by means of a gas anesthesia machine (2.5% isoflurane to induce anesthesia, 1.5% to maintain anesthesia through a nose-mask). NPY (0.2 μL, 234 μM) was dissolved in ACSF and administered through a cannula with a 1 μL Hamilton syringe connected with polyethylene tubing to a 13.1 mm stainless steel injector cannula (30 gauge). NPY Y5 receptor antagonist (0.6 ml, 10 mg/kg) was dissolved in 32% 2-hydroxypropyl-B-cyclodextrin, and injected s.c. 30 minutes before NPY and/or light stimulation. Light pulses (5 min, 150 lux) were delivered individually by placing animals under two white fluorescent tubes (Phillips, model F30T12); the timing of the light pulses was selected to be in the animals' dark period, 3.5 hours before lights would normally come on.

Animals were allowed at least 10 days under LD (14 hours of light, 10 hours of dark) in order to establish a stable rhythm, and then housed under constant dim red light (DRL) provided by a safelight lamp (Coastar, Inc. <1 lux). Two sets of experiments comprising five treatments were delivered in a counterbalanced design: NPY alone, NPY+light; light alone, light+NPY Y5 antagonist, NPY+NPY Y5 antagonist+light. After two treatments (only one of them involving light stimulation), animals were resynchronized to the previous LD cycle for 7-10 days, and then exposed again to dim red light for the second set of treatments. In this way, the animals did not spend more than 3 weeks under dim red light, and did not receive more than one light pulse or more than 4 treatments overall.

Data analysis. For in vivo experiments, data were automatically collected and analyzed with Clocklab software bundle (ActiMetrics Software, Evanston, Ill.). Two investigators blind to the treatment analyzed phase advance magnitudes. Statistical analyses were performed by means of ANOVA followed by Student-Newman-Keul's test.

Results. The NPY Y5 receptor antagonist of Formula IIa was selected for all in vivo studies. Briefly, treatments administered were: Light, NPY, Light+NPY, Light+NPY+NPY Y5 receptor antagonist, Light+NPY Y5 receptor antagonist and NPY Y5 receptor antagonist alone. As shown in Table 2, results show that NPY significantly blocked the light induced phase advance and the NPY Y5 antagonist significantly reversed this blockade. Furthermore, the NPY Y5 antagonist potentiated the phase shift induced by light when applied alone, 30 min before light stimulation. Neither the NPY Y5 antagonist applied alone, nor NPY or the combination of both induced any change in the phase of the wheel running rhythms in absence of light stimulation at that circadian time.

Taken together, these results support the conclusion that the NPY Y5 antagonist of fomula IIa robustly blocks the effects of NPY when it is given exogenously through the cannula. The NPY Y5 antagonist of formula IIa also blocks the effects of endogenous NPY as is indicated by its ability to enhance the natural ability of light to produce phase advances.

TABLE 2
EFFECTS OF NPY Y5 ANTAGONISTS ON LIGHT-INDUCED PHASE
ADVANCES OF HAMSTER WHEEL RUNNING ACTIVITY

| Treatment | | Phase shift (h) |
|---|---|---|
| a. | Light | 1.33 ± 0.10 |
| b. | Formula IIa + light | 2.11 ± 0.16 |
| c. | NPY + light | −0.03 ± 0.20 |
| d. | Formula IIa + NPY + light | 1.18 ± 0.27 |
| e. | Formula IIa | −0.12 ± 0.04 |
| f. | NPY | −0.04 ± 0.11 |
| g. | Formula IIa + NPY | −0.20 ± 0.07 |

Experimental Conditions:

Phase advances (h) were calculated as the difference in the onset of running behavior in animals kept in dim red light relative to those exposed to combination of light and/or drug treatments. Means±S.E.M. for N=7-12.
a. Light alone experiment in which the animals are exposed to light 3 hours before the scheduled beginning of the animals' period of normal light. There is a resulting phase advance of 1.33 hours.
b. Formula IIa+light experiment in which the animals are pretreated with NPY Y5 antagonist of formula IIa and then exposed to light 3 hours before the scheduled beginning of the animals' period of normal light. The dose of compound of formula IIa is 10 mg/kg s.c. given 30 minutes prior to light exposure. There is an enhancement of the light-induced phase advance by compound of formula IIa to 160% of the light alone experiment.
c. NPY+light experiment in which the animals are pretreated with NPY and then exposed to light 3 hours before the scheduled beginning of the animals' period of normal light. The dose of NPY is 200 ng/nl in a volume of 0.2 μL delivered by syringe into a cannula placed adjacent to the SCN. There is a complete blockade of the phase advance compared to that produced in the light alone experiment.
d. Formula IIa+NPY+light experiment in which the animals are pretreated with NPY and NPY Y5 antagonist of formula IIa and then exposed to light 3 hours before the scheduled beginning of the animals' period of normal light. The dose of NPY is 200 ng/nl in a volume of 0.2 μL delivered by syringe into a cannula placed adjacent to the SCN immediately before exposure to light. The dose of compound of formula IIa is 10 mg/kg s.c. 30 minutes prior to light exposure. There is a significant reversal of the effects of NPY on light-induced phase advances by compound of formula IIa to 89% of the light alone experiment.
e. Formula IIa alone experiment in which the animals are given NPY Y5 antagonist of formula IIa alone. The dose of compound of formula IIa is 10 mg/kg s.c. given 3.5 hours before the scheduled beginning of the animals' period of normal light. There is no effect on the phase of wheel running activity.
f. NPY alone experiment in which the animals are given NPY alone. The dose of NPY is 200 ng/nl in a volume of 0.2 μL delivered by syringe into a cannula placed adjacent to the SCN. There is no effect on the phase of wheel running activity.
g. formula IIa+NPY experiment in which the animals are treated with NPY and NPY Y5 antagonist of formula IIa 3 hours before the scheduled beginning of the animals' period of normal light. The dose of NPY is 200 ng/nl in a volume of 0.2 μL delivered by syringe into a cannula placed adjacent to the SCN. The dose of compound of formula IIa is 10 mg/kg s.c. given 30 minutes prior to NPY. There is no effect on the phase of wheel running activity.

1. A method of modulating circadian rhythm responses to light in a mammal by administering to a mammal an amount of an NPY Y5 receptor antagonist effective in modulating circadian rhythm responses to light.

2. A method for enhancing the effects of light on circadian rhythm in a mammal by administering a light enhancing amount of an NPY Y5 receptor antagonist to a mammal including humans.

3. A method according to claim 1 or 2 wherein the NPY Y5 antagonist is a compound of the formula 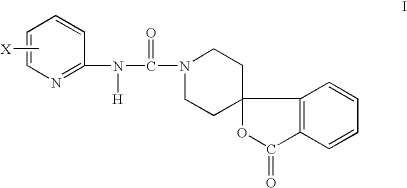
wherein X is selected from the group consisting of chlorine, bromine, fluorine, iodine, trifluoromethyl, hydrogen, cyano, C₁ to C₆ alkyl, C₁ to C₆ alkoxy, C₅ or C₆ cycloalkyl, ester, amido, aryl and heteroaryl.

4. A method according to claim 3, wherein the NPY Y5 antagonist is a compound of the formula

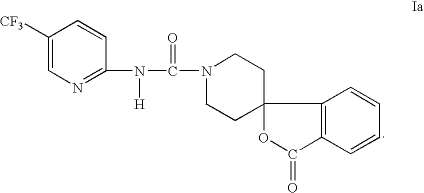

5. A method according to claim 1 or 2 wherein the NPY Y5 antagonist is a compound of the formula 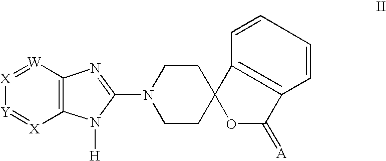
or a pharmaceutically acceptable salt, solvate or prodrug thereof or any of the foregoing; wherein A is oxygen or hydrogen; W, X, Y and Z are independently N or CR₁ wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono- or di-(C₁-C₆)alkylamino or (C₁-C₆)alkoxy, (C₃-C₇)cycloalkyl, (C₃-C₇)cycloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono- and di(C₁-C₆)alkylamino(C₁-C₆)alkyl.

6. A method according to claim 5, wherein the NPY Y5 antagonist is a compound of the formula

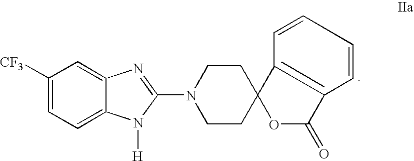

7. A method of treating circadian rhythm disorders in mammals including humans comprising administering to a mammal an amount of an NPY Y5 antagonist effective in treating circadian rhythm disorders.

8. A method of treating circadian rhythm disorders in mammals including humans comprising administering to a mammal a light enhancing amount of an NPY Y5 antagonist effective in treating circadian rhythm disorders.

9. A method according to claim 7 or 8 wherein said disorder is associated with NPY blockades of light induced circadian phase advances.

10. A method according to claim 9 wherein said NPY blockade is reversed by said NPY Y5 antagonist.

11. A method according to claim 7 or 8 wherein the NPY Y5 antagonist is a compound of the formula 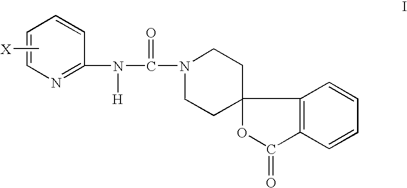
wherein X is selected from the group consisting of chlorine, bromine, fluorine, iodine, trifluoromethyl, hydrogen, cyano, C₁ to C₆ alkyl, C₁ to C₆ alkoxy, C₅ or C₆ cycloalkyl, ester, amido, aryl and heteroaryl.

12. A method according to claim 11, wherein the NPY Y5 antagonist is a compound of the formula

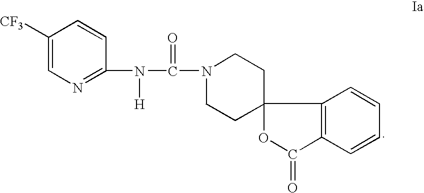

13. A method according to claim 7 or 8 wherein the NPY Y5 antagonist is a compound of the formula 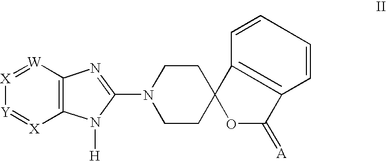
or a pharmaceutically acceptable salt, solvate or prodrug thereof or any of the foregoing; wherein A is oxygen or hydrogen; W, X, Y and Z are independently N or CR, wherein R₁ is independently selected at each occurrence from hydrogen, halogen, hydroxy, nitro, cyano, amino, (C₁-C₆)alkyl, (C₁-C₆)alkoxy, (C₁-C₆)alkoxy substituted with amino, mono- or di-(C₁-C₆)alkylamino or (C₁-C₆)alkoxy, (C₃-C₇)cycloalkyl, (C₃-C₇)cycloalkyl(C₁-C₄)alkyl, (C₂-C₆)alkenyl, (C₃-C₇)cycloalkenyl, (C₂-C₆)alkynyl, (C₃-C₇)cycloalkynyl, halo(C₁-C₆)alkyl, halo(C₁-C₆)alkyl, halo(C₁-C₆)alkoxy, mono and di(C₁-C₆)alkylamino, amino(C₁-C₆)alkyl, and mono- and di(C₁-C₆)alkylamino(C₁-C₆)alkyl.

14. A method according to claim 13, wherein the NPY Y5 antagonist is a compound of the formula

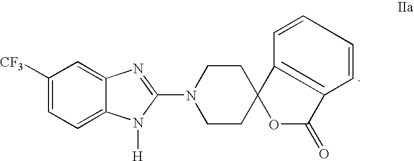

15. A method according to claim 7 or 8, wherein the NPY Y5 antagonist is administered to a mammal prior to experiencing circadian rhythm disorders.

16. A method according to claim 7 or 8, wherein the NPY Y5 antagonist is administered to a mammal predisposed to or at risk of experiencing circadian rhythm disorders.

17. A method according to claim 1, wherein said modulation comprises reversing NPY caused blockade by the administration of an NPY-Y5 antagonist of Formula I or Formula II.

18. A method according to claim 17, wherein said NPY-Y5 antagonist of Formula 1 is the compound of Formula Ia wherein said compound of Formula Ia exhibits, in vitro, about 70% reversal of the blockade caused by NPY.

19. A method according to claim 18, wherein said NPY-Y5 antagonist is a compound of Formula ha wherein said compound exhibits, in vitro, about 95% reversal of the blockade cause by NPY.

20. A method according to claim 9 wherein said compound of Formula ha exhibits, in vivo, about 90% reversal of the blockade caused by NPY.

21. A method according to claim 5 wherein said NPY Y5 antagonist is a compound of formula IIa which in the absence of NPY enhances an in vivo light induced phase shift by 160% of that achieved by light alone.

22. A method of reversing the effect of NPY on the light induced phase advances in a mammal comprising administering to said mammal a effective amount of a compound of Formula I or Formula II to reverse the effect of NPY.

23. A method of treating circadian rhythm phase disorders comprising administrating to a mammal in need of such treatment a therapeutically effective amount of a compound which provides a blockade of at least 70% to NPY Y5 receptors.

24. A method according to claim 23 wherein said compound is selected from the group consisting of compounds of Formula I and Formula II.

25. A method according to claim 24 wherein said compound is selected from the group consisting of the compound of Formula Ia and the compound of Formula IIa.

26. A method of treating a circadian rhythm disorder selected from disorders of phase related to jet lag and shift work, depression, bipolar disorder, seasonal affective disorder, anxiety, schizophrenia, Alzheimers Disease, rapid eye movement (REM) sleep disorders, advanced sleep phase syndrome, delayed sleep phase syndrome, non-24-hour sleep-wake disorder, hypersomnia, parasomnia, narcolepsy, nocturnal enuresis, obesity and restless-leg syndrome comprising administering to a mammal in need of such treatment a therapeutically effective amount of the compound of formula I.

27. A method of treating a circadian rhythm disorder selected from disorders of phase related to jet lag and shift work, anxiety, depression, bipolar disorder, seasonal affective disorder, schizophrenia, Alzheimers Disease, rapid eye movement (REM) sleep disorders, advanced sleep phase syndrome, delayed sleep phase syndrome, non-24-hour sleep-wake disorder, hypersomnia, parasomnia, narcolepsy, nocturnal enuresis, obesity and restless-leg syndrome comprising administering to a mammal in need of such treatment a therapeutically effective amount of the compound of formula II.

28. A method according to claim 26 or 27 wherein the depression disorder is selected from the group consisting of unipolar depression, bipolar depression, seasonal affective disorder and dysthymia.

29. A method according to claim 1 wherein said modulation of circadian rhythm responses to light comprises phase-shifting, resetting of the circadian clock and enhancing the rate of re-entrainment.

30. A method according to claim 2 wherein said NPY Y5 antagonist enhances an in vivo light induced phase shift by 200% of that achieved by light alone.

31. A method according to claim 7 or 8 wherein said circadian rhythm disorders are comprised of circadian rhythm phase-shift disorders.

32. A method according to claim 31 wherein said circadian rhythm phase-shift disorders include a phase-shift advance or a phase-shift delay.

33. A method according to claim 7 or 8 wherein said circadian rhythm disorders are comprised of changes in the amplitude of the circadian rhythm.
